(19) United States
(12) Patent Application Publication
(10) Pub. No.: US 2022/0025326 A1
(43) Pub. Date: Jan. 27, 2022

(54) KITS AND METHOD OF MODIFYING, DETECTING, AND SORTING ANTIBODY-PRODUCING CELLS

(71) Applicant: Namocell Inc., Mountain View, CA (US)

(72) Inventor: Junyu LIN, Palo Alto, CA (US)

(21) Appl. No.: 17/443,415

(22) Filed: Jul. 26, 2021

Related U.S. Application Data
(60) Provisional application No. 63/057,232, filed on Jul. 27, 2020.

(51) Int. Cl. C12N 5/0781

(52) U.S. Cl. CPC C12N 5/0635; C12N 2500/36; C12N 2501/051; C12N 2500/50

(57) ABSTRACT

Described herein are methods and compositions for modifying a mammalian cell plasma membrane to retain secreted antibodies, capturing antibodies produced by the cells and sorting antibody producing cells according to antibody specificities or antibody quantities. These methods and compositions may be particularly useful for biological and medical applications. The methods may include modifying cell membranes from a population of antibody producing cells by inserting a membrane anchor into the cell membranes, a generic antibody capture molecule attached to the cell anchor, capturing antibodies produced by the cell, and detecting and dispending cells according to antibody specificities or antibody quantities with a microparticle sorting and dispensing apparatus.

### CROSS REFERENCE TO RELATED APPLICATIONS

This patent application claims priority to U.S. Provisional Patent Application No. 63/057,232, titled “KITS AND METHOD OF MODIFYING, DETECTING, AND SORTING ANTIBODY-PRODUCING CELLS” and filed on Jul. 27, 2020, herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are methods and compositions (including kits) capturing antibody producing cells that include modifying a cell plasma membrane to retain antibodies secreted by the cell. These methods and compositions may be part of a cell and single-cell capture method and system. These methods and compositions may be particularly useful for biological and medical applications.

### BACKGROUND

Antibodies are widely used for performing many types of biological and medical research. Challenges to performing research with antibodies include having the right antibody to study and obtaining enough of the antibodies to perform the research. While antibodies are made inside cells, they are typically secreted outside of the cell to act on foreign antigens or pathogens. Monoclonal antibody development (including hybridoma development) can be costly and time consuming, particularly in characterizing single cells that produce high titers of monoclonal antibodies having desirable binding properties. Currently, this is done by manually or semi-automatically culturing individual cells and assaying the secreted antibody in the supernatant; this process is a time consuming and labor-intensive process. The number of cells that can be assayed is limited. It may be important to measure how much protein a mammalian cell can secrete over a period of time, particularly when a mammalian cell is used to produce a protein. For example, Chinese hamster ovary (CHO) cells are used commercially in the production of therapeutic proteins. To increase the production yield of the therapeutic protein, a single CHO cell producing the highest amount of the therapeutic protein is selected from amount thousands of cells. Currently, most common ways to select the highest producer cell are by measuring the amount of secreted protein in medium from clonal cell culture, or by measuring halo size and intensity of secreted protein from cell clones grown on semi-solid agar. There are no direct ways to measure the amount of a given secreted protein each individual CHO cell can produce over a period of time in the liquid culture medium. Further, sorting individual cells from larger mixtures of cells is difficult and time-consuming.

Antibodies are secreted into cell culture medium and not retained on the plasma membrane. A universal method to trap secreted antibodies on the surface of the cell which is producing the antibodies and sort these cells based on specificities of surface-bound antibodies or the quantities of surface-bound antibodies would be valuable.

### SUMMARY OF THE DISCLOSURE

Described herein are methods and compositions (e.g., kits) for presenting antibodies produced by a cell (e.g., a mammalian cell) on the outer cell membrane surface of the cell producing the cell, and sorting these cells efficiently and directly based on this presentation. These methods may allow processing of mixtures of cells, which may be more efficiently cultured and may allow pools of antibodies to be assayed quickly prior to sorting, enhancing the speed and efficacy of antibody production and characterization. By attaching produced antibodies on the surface of the cells producing them, the cells which secrete antigen specific antibodies may be identified by labeling the cells (e.g., with a fluorescence marker); for example by using a labeled antigen specific to the produced antibody, and/or labeling an aptamer or antibody that binds to the produced antibody, or to a complex including the antibody produced by the cell and the agent binding it to the cell surface. Trapping antibodies on the cell surface could also be used to identify cells which can secrete antibodies at a higher rate by measuring the amount of the antibodies on the cell surface over a period of time.

It is known that protein A and protein G bind to a variety of different categories of antibodies at high affinity (Grodzki A. C. Methods Mol Biol. 2010; 588:33-41). Protein A is a 42 kDa surface protein originally found in the cell wall of the bacteria Staphylococcus aureus. Protein G is an immunoglobulin-binding protein expressed in group C and G Streptococcal bacteria, much like Protein A but with differing binding specificities. Described herein are methods and compositions including a soluble construct that includes both protein A and protein G binding domains that can be added to cell media to be anchored on the plasma membrane of the cells, e.g., cells secreting antibodies. As shown herein, antibodies secreted out from these cells binds to protein A or protein G binding domains and is trapped on the cell surface. Surprisingly, the antibodies are captured in sufficient amounts to be detectable. Although previous work has demonstrated that palmitate-conjugated protein A can coat cell plasma membranes when added into a cell culture medium (Kim S. A. J Immunol Methods. 1993 Jan. 14; 158(1):57-65.), the low solubility of palmitate in water required that detergent be used to increase the solubility of palmitate-conjugated protein A, resulting in an unacceptable level of cellular toxicity.

Described herein are constructs including a protein A and/or protein G function domains that are conjugated to an oleyl chain and poly(ethylene glycol) (PEG) derivative. The oleyl chain may anchor proteins into the plasma membrane of mammalian cells, while being soluble in cell media. Specifically, oleic acid-PEGs is water soluble and not toxic to the cells.

Recently, Li developed a rapid method to isolate antigen-specific hybridoma (Li X. Anal Chem. 2018 Feb. 6; 90(3):2224-2229) by covalently linking an oleic acid PEG to an antigen, which may be used to capture secreted antibody. However, to capture a different antibody, a different oleic acid PEG conjugated antigen would have to be made for each antibody, a costly and inconvenient process and some antigens might not get conjugated. Kida covalently linked an oleic acid PEG to a capture antibody, which can bind to secreted antibodies (Anal Chem. 2013 Feb. 5; 85(3):1753-9). In this method, the capture antibody can bind to antibodies from one species. However, it cannot bind to antibodies from a different species. Also, multiple steps were required to capture and detect secreted antibodies on the cell surface. It would be particularly useful to have a simple method which can capture and detect any kind of antibodies on any kind of cell surface. Described herein are techniques and compositions that can address this and other challenges for antibody production and isolation.

For example, described herein are methods of capturing an antibody producing cell that include: contacting a population of antibody-producing cells with a soluble presentation construct comprising a lipid modified protein A molecule (MPA), lipid modified protein G molecule (MPG) or a lipid modified protein A/G molecule (MPAG) so that the MPA, MPG or MPAG is presented on the surface of the antibody-producing cells; incubating the antibody-producing cells so that antibody produced by the cells is captured by the MPA, MPG or MPAG on the surface of the antibody-producing cells for less than 24 hours (e.g., less than 20 hours, less than 18 hours, less than 16 hours, less than 12 hours, less than 10 hours, less than 8 hours, less than 6 hours, less than 5 hours, less than 4 hours, less than 3 hours, less than 2 hours, etc.); exposing the antibody-producing cells to a detection agent comprising a detectably labeled protein A molecule, a detectably labeled protein G molecule and/or a detectably labeled protein A/G molecule; sorting the cells using a cell sorting device according to the level of the detection agent.

The detection agent may comprise, for example, a fluorescently-labeled antigen, a fluorescently-labeled protein A, fluorescently-labeled protein G, and/or a fluorescently-labeled protein A/G. The detection agent may be matched to the presentation construct, so that if the presentation construct is a protein A/G construct, then the detection agent may be a protein A/G construct (without the lipid modification). For example, the detection agent may comprise a fluorescently-labeled protein A/G (FPAG), and the presentation construct may be a lipid-modified protein A/G (MPAG).

In general, the concentration of the lipid-modified soluble presentation construct may be chosen so that it does not saturate. For example, the lipid-modified soluble presentation construct may be present at a concentration of less than x uM (e.g., where x is about 100, 10, 1, 0.5, 0.1, 0.05, 0.01, etc.).

In general, the MPA, MPG and/or MPAG may comprise a fatty acid based chain attached to a solubilizer. In some variations the MPA, MPG and/or MPAG comprises at least two fatty acid based chains attached to a solubilizer. For example, the MPA, MPG and/or MPAG comprises at least two omega-9 fatty acid based chains attached to a solubilizer. The MPA, MPG and/or MPAG may comprise at least two oleic acid based chains attached to a solubilizer. The solubilizer may comprise an attached poly(ethylene glycol) (PEG) polymer.

Any appropriate antibody-producing cells may be used. For example, the step of contacting a population of antibody-producing cells may comprise contacting a hybridoma, contacting a population of Chinese Hamster Ovary (CHO) cells, contacting a population of antibody-producing B cells, and/or contacting a population of antibody-producing plasma cells.

The soluble presentation construct may consist essentially of protein A/G. In some variations, the soluble presentation construct consists essentially of protein A. In some variations, the soluble presentation construct consists essentially of protein G.

The step of exposing the antibody-producing cells to the detection agent may comprise exposing the antibody-producing cells to a detection agent having a detectably labeled marker comprising a fluorescently labeled antigen.

For example, described herein are methods of capturing an antibody producing cell, the method comprising: contacting a population of antibody-producing cells with a soluble presentation construct comprising a lipid modified protein A/G molecule (MPAG) having at least two oleic acid based chains coupled to a poly(ethylene glycol) (PEG) polymer that is coupled to the protein A/G molecule, so that the MPAG is presented on the surface of the antibody-producing cells; incubating the antibody-producing cells so that antibody produced by a cell in the population of antibody-producing cells is captured by the MPAG on the surface of that cell; exposing the antibody-producing cells to a detection agent detection agent comprising a detectably labeled marker that binds to the antibody produced by the cells; sorting the cells using a cell sorting device according to the level of the detection agent. The detection agent may comprise an antigen of interest coupled to the labeled marker, wherein the antibody binds to the antigen of interest. For example, the detection agent may comprise protein A/G coupled to a florescent marker. The step of incubating may comprise incubating for between 1 minutes and 24 hours (e.g., 22 hours or less, 20 hours or less, 18 hours or less, 16 hours or less, 14 hours or less, 12 hours or less, 10 hours or less, 8 hours or less, 6 hours or less, 5 hours or less, 4 hours or less, 3 hours or less, 2 hours or less, etc.).

A kit for isolating antibody-producing cells may include: a water-soluble presentation agent comprising a solution of a lipid modified protein A/G molecule (MPAG) having at least two oleic acid based chains coupled to a poly(ethylene glycol) (PEG) polymer that is coupled to the protein A/G molecule; and a detection agent comprising a solution of fluorescently labeled protein A/G. In some variations, a kit for isolating antibody-producing cells may include: a water-soluble presentation agent comprising a solution of a lipid modified protein A/G molecule (MPAG) having at least two oleic acid based chains coupled to a poly(ethylene glycol) (PEG) polymer that is coupled to the protein A/G molecule; and a composition for fluorescently labeling an antigen (e.g., a protein). The composition may be configured to add one or more florophores (e.g., FITC) to a protein. For example, a kit may include reagents for attaching a fluorophore to a protein antigen using a first reactive group; a complementary reactive group may be attached to the antigen protein for labeling. In some variations, the kit may include reagents for making an antigen protein florescent by reaction with a maleimide-conjugated fluorophore and cysteine residue on the antigen protein. In some variations the antigen protein may be reacted with a succinimidyl-ester conjugated fluorophore and N-terminal amine on the antigen protein. In some variations the kit includes reagents for ligating a fluorescent peptide to a thioester on the antigen of the protein through an N-terminal cysteine residue. In some variations, the kit may include a self-labeling protein tag, the SNAP-Tag and reagents for processing; a fluorescent O⁶-benzylguanine may be cleaved by hAGT resulting in the fluorophore being covalently linked to the hAGT and the protein antigen. In some of the kits described herein, the kit may include the reagents for biotinylation of the antigen protein at a biotin recognition sequence (BRS) by BirA and conjugation with streptavidin-fluorophore or functionalized quantum dot.

The protein A, protein G and/or protein A/G constructs described herein may be recombinant fusion proteins. For example, the protein A/G constructs described herein combine IgG binding domains of both Protein A and Protein G, and are referred to as “protein A/G.” Protein A/G contains four Fc binding domains from Protein A and two from Protein G, yielding a final mass of about 50,460 Daltons. Protein A/G has the additive properties of Protein A and G for binding to antibodies.

As described herein, protein A, protein G and/or protein A/G were covalently linked to an oleyl chain and poly(ethylene glycol) derivative. Modified protein A, protein G and/or protein A/G can be easily incorporated into cell plasmas membrane and bind to secreted antibodies from the cell. Thus, to detect the cell surfaced bound antibody (e.g. presented on the outer surface of the cell), a presentation construct, which may be a soluble membrane-binding protein A/G construct (or in some variations modified protein A construct or modified protein G construct) may be included in the cell media to couple secreted antibodies to the cell surface. The presentation construct may be referred to herein as a modified protein A/G molecule (or MPAG), a modified protein A construct (MPA), or a modified protein G construct (MPG), also referred to herein as a lipid-modified protein A/G molecule, lipid-modified protein A construct, or lipid-modified protein G construct. Thereafter a detection agent may be used to detect the bound antibody. In particular, the detection agent may be, e.g., a fluorescently-labeled antigen, or a fluorescently-labeled protein A/G construct (FPAG), such FITC-labeled protein A/G construct), or a fluorescently-labeled protein A construct (FPA), or a fluorescently-labeled protein G construct (PFG).

The detection or cell isolation system may include a presentation construct (e.g., an oleyl chain and poly(ethylene glycol) derivative coupled to a protein A/G construct). By modifying a single protein: protein A/G, antibodies secreted from a cell can be captured, detected and sorted.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A shows one example of a soluble presentation construct, including a lipid modified protein A/G molecule (MPAG).

FIG. 1B schematically illustrates the lipid modified protein A/G molecule (MPAG) from FIG. 1A anchored to a cell. In practice multiple MPAGs may be coupled to the cells(s). and may bind to released antibody produced by the cell.

FIG. 1C schematically illustrates a lipid modified protein A/G molecule (MPAG) from FIG. 1A anchored to a cell and capturing antibodies secreted from the cell.

FIG. 2A is a diagram showing an example of secreted antibodies that are captured by MPAG being detected by an FITC conjugated antigen (e.g., using an antigen-specific detection agent including the antigen).

FIG. 2B is an example, similar to that shown in FIG. 2A, in which the antigen of interest is not recognized or appreciably bound by the captured (and presented) antibody secreted from the cell.

FIG. 2C is a diagram illustrating one example of a secreted that is captured by cell-surface MPAG and is detected by a generic detection agent, shown here as an FITC conjugated protein A/G (FPAG) construct.

FIG. 3A shows a picture of CHO cells treated with MPAG and stained with FITC labeled mouse anti-human IgG antibody. FIG. 3B shows a picture of untreated CHO cells stained with FITC labeled mouse anti-human IgG antibody only.

FIG. 4A shows results of an analysis of the fluorescent intensity of CHO cells treated with MPAG and stained with FITC labeled mouse anti-human IgG antibody. FIG. 4B shows an analysis of the fluorescent intensity of CHO cells stained with FITC labeled mouse anti-human IgG antibody only.

FIG. 5 shows results of an analysis of fluorescent intensity of CHO cells treated with MPAG and stained with FITC labeled mouse anti-human IgG antibody over time.

FIG. 6A shows the results of an analysis of CHO cells secreting a human IgG treated with MPAG and stained with FITC conjugated protein A/G (FPAG). FIG. 6B shows the results of an analysis of CHO cells secreting a human IgG and treated with FPAG only.

FIG. 7 shows examples of fatty acids that can be used as membrane anchors.

FIG. 8A schematically illustrates one example of a system for cell sorting that may be used as described herein.

FIG. 8B illustrates one example of a flow switch that may be used with the system of FIG. 8A for sorting cells labeled as described herein.

### DETAILED DESCRIPTION

Described herein are methods and compositions for modifying, detecting, and sorting antibody-producing cells of interest from a population of antibody producing cells. These methods may utilize a generic antibody capture molecule to capture antibodies secreted from the same cell. These methods allow large pools of antibody producing cells to be interrogated for desired characteristics, such as particular antibody binding specificities or antibody production levels. In particular, these methods and compositions allow the cells in the pools to be individually interrogated and individual antibody producing cells of interest to be sorted and isolated. These methods and compositions do not require a multiple different antibody capture molecules to be manufactured for interrogating different antibody populations (e.g., from different species or different classes).

For example, a method of capturing an antibody producing cell may include modifying cell membranes from a population of antibody producing cells by inserting a membrane anchor into the cell membranes, a generic antibody capture molecule attached to the membrane anchor. The method may include producing antibodies from the cell population and capturing produced antibodies with the generic antibody capture molecule from the same cell. The method may include exposing the cells to a detectably labeled marker that binds to an antibody of interest and binding the detectably labeled marker to the antibody of interest. The method may include delivering the population of antibody producing cells to a microparticle sorting and dispensing apparatus configured to analyze single cells, detecting the detectably labeled marker from a cell. The method may include dispensing, with the microparticle sorting and dispensing apparatus, cells having a threshold level of detectably labeled marker into a sample outlet flow path, and dispensing cells not having a threshold level of detectably labeled marker into a waste outlet flow path.

### Definitions

Protein A originally derived from a cell wall anchored protein in the bacteria Staphylococcus aureus. Protein A binds to antibodies (immunoglobulin G (IgG), immunoglobulin M (IgM), immunoglobulin E (IgE), and immunoglobulin A (IgA)) at their conserved regions. Protein A binds to antibodies from a variety of species including human, cat, dog, pig, rabbit, and mouse.

Protein G originally derived from the bacteria Streptococci. Protein G binds most mammalian immunoglobulin G (IgG), including human IgG3, rat and mouse IgG2a, and rabbit IgG. It does not substantially bind to human IgM, IgD and IgA. Protein G binds to antibodies has from a variety of species including human, bovine, goat, guinea pig, horse, mouse, pig, rabbit, and sheep.

Protein A/G refers to a recombinant fusion protein that combines parts of the binding domains from both protein A and protein G. Protein A/G can bind to antibody species and subclasses recognized by either Protein A or Protein G as well as to some additional antibodies. Protein A/G binds to IgG and can also bind to human IgA, IgE, IgM, and IgD. Protein A/G binds to antibodies has from a variety of species including human, bovine, cat, dog, goat, guinea pig, horse, mouse, pig, rabbit, and sheep.

Lipid modified protein A/G molecule (MPAG) is modified protein A/G including a fatty acid based chain attached to a solubilizer. In some variations MPAG includes two or more oleic acid based chains, linked to poly(ethylene glycol), and to protein A/G.

Jacalin is plant-based lectin with a molecular weight of 66 kDa originally derived from Artocarpus integrifolia (jackfruit). Jacalin binds to some immunoglobulins and can be used as an antibody capture molecule.

A generic antibody capture molecule refers to a molecule that can bind to multiple different or classes of antibodies and/or antibodies from multiple animal species, such as by binding to the Fc or constant region.

An antibody refers to a whole antibody or a fragment thereof. An antibody can be intact, chimeric (e.g., made from domains from different species) or otherwise modified (e.g., humanized). An intact antibody is an immunoglobulin molecule having four polypeptide chains, two heavy (H) chains and two light (L) chains inter-connected by disulfide bonds. Each heavy chain has a heavy chain variable region (abbreviated herein as HCVR or VH) and a heavy chain constant region (CH). The heavy chain constant region has three domains, CH1, CH2 and CH3. Each light chain includes a light chain variable region (abbreviated herein as LCVR or VL) and a light chain constant region. The light chain constant region includes one domain, CL. The VH and VL regions can be further subdivided into regions of hypervariability, termed complementarity determining regions (CDRs), interspersed with regions that are more conserved, termed framework regions (FR). Each VH and VL has three CDRs and four FRs, arranged from amino-terminus to carboxy-terminus in the following order: FR1, CDR1, FR2, CDR2, FR3, CDR3, FR4. Antibodies that can be successfully assayed using the methods described herein include, but are not limited to, human, cat, chicken, cow, dog, donkey, goat, guinea pig, hamster, horse, koala, llama, monkey, mouse, pig, rabbit, rat, or sheep. Antibodies that can be successfully assayed using the methods described herein include, but are not limited to, IgA, IgA1, IgA2, IgD, IgE, IgG, IgG1, IgG2, IgG3, IgG4, and IgM antibodies.

Antibody producing cells includes cells that naturally produce antibodies (such as immune B cells) as well as those altered to produce antibodies, such as hybridomas or by cell transfection. Cells of interest include CHO cells as well as NSO, Sp2/0, HEK293, and PER.C6.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species, but such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “detectably labelled” when used herein refers to a detectable compound or composition that is conjugated directly or indirectly to a compound to generate a “labeled” compound. The label may be detectable by itself (e.g. radioisotope labels or fluorescent labels) or, may catalyze chemical alteration of a substrate compound or composition that is detectable (e.g., avidin-biotin) in the case of an enzymatic label. A detectable label can be an enzyme, radioisotope, a fluorochome, a luminescent tag, a metal, or a dye.

A detection agent, as used herein, may be a specific detection agent or a generic detection agent. The specific detection agent may include an antigen of interest coupled to a labeled marker (e.g., a florescent marker); the antigen of interest typically binds to the antibody that is the target of the process. The generic detection agent may include protein A/G coupled to a labeled marker (e.g., a florescent marker).

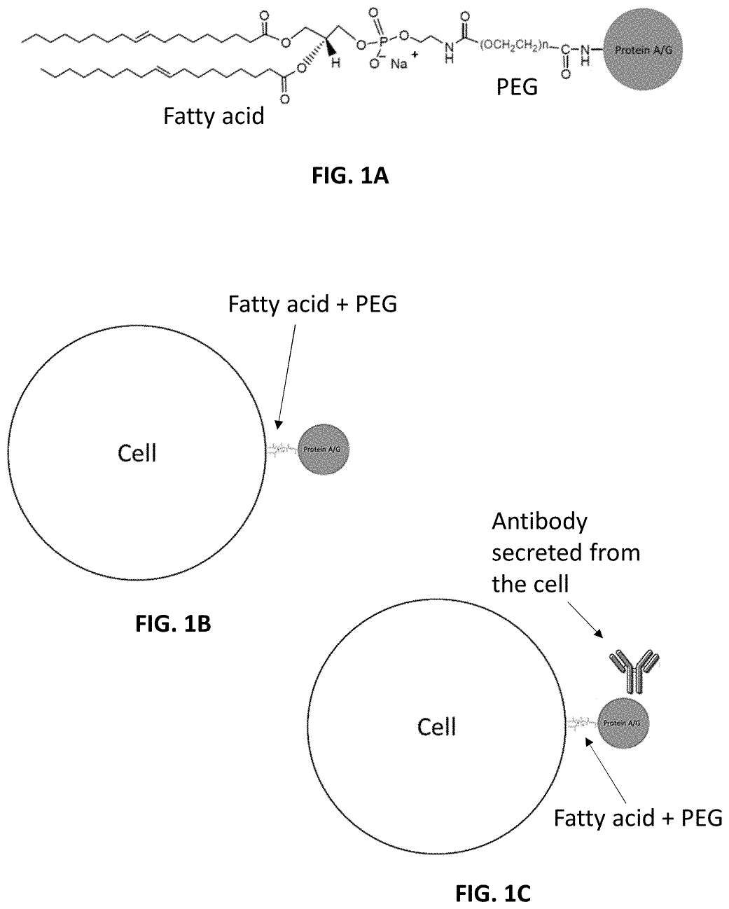

Fatty acids are carboxylic acids with a long aliphatic chain. Fatty acids include a linear unsaturated fatty acid having 12 to 30 carbon atoms such as palmitoleic acid, oleic acid, linoleic acid, linolenic acid, and arachidonic acid. Fatty acids include a linear saturated fatty acid having 16 to 30 carbon atoms such as palmitic acid, stearic acid, arachidic acid, behenic acid, lignoceric acid, and montanic acid. FIG. 1 and FIG. 7 show examples of fatty acids. A fatty acid of interest is oleic acid.

Poly(ethylene glycol) (PEG) refers to a polyether polymer of repeating ethyleneoxy units, H—(O—CH2-CH2)ₙ—OH where n refers to the number of repeats. PEG is hydrophilic, water-soluble, non-toxic, and biocompatible. PEG can be referred to by its approximate molecular weight, which depends on the number of ethyleneoxy repeats. For example, PEG600 is a PEG polymer with a molecular weight (MW) of 600 and PEG10K is a PEG polymer with a molecular weight of 10K or 10,000 g/mol.

A solubilizer is a compound that helps solubilize or dissolve a fatty acid and generic antibody capture molecule in cell culture media.

### DESCRIPTION

In general, described herein are methods and kits for isolating one or more single cells producing a target antibody (e.g., an antibody against a particular antigen having a desired specificity and/or specific functions, such neutralizing activities or inhibitory activities) from a population of cells (including a population of antibody-producing cells, such as hybridoma cells, B cells or plasma cells). A method of capturing an antibody producing cell may include modifying cell membranes from a population of antibody producing cells by inserting a membrane anchor into the cell membranes, a generic antibody capture molecule attached to the membrane anchor. A membrane anchor may include one or more than one fatty acid based chains (e.g., two) attached to a solubilizer. Cell membranes are hydrophobic and fatty acids are part of cell membranes. While a fatty acid portion of a membrane anchor may insert into a cell membrane, fatty acids might not readily dissolve in cell media containing the cells.

A solubilizer is a compound that helps solubilize or dissolve the fatty acid and generic antibody capture molecule in cell culture media. A solubilizer may be covalently or non-covalently attached to a fatty acid to aid in solubilizing or dissolving the membrane anchor in cell media. A solubilizer may be attached to a fatty acid anywhere. In some examples, a solubilizer is attached to one or more than one fatty acid at its acid moiety, either directly or via a linker. A solubilizer may be a compound that readily dissolves in an aqueous solution (such as cell media) and is of appropriate size and composition to help dissolve or solubilize the fatty acid. A solublizer may be a water-soluble polymer (e.g., poly(alkylene oxide), poly(ethylene glycol, poly(vinyl pyrrolidone), poly(vinyl alcohol). A water-soluble polymer of interest is poly(ethylene glycol) (PEG) as it is non-toxic to cells and has been used in cell media.

FIG. 1A shows a membrane anchor with a solubilizer and generic antibody capture molecule. FIG. 1A shows protein A/G covalently linked to oleic acid PEG. The molecular weight of the oleic acid PEG portion in FIG. 1A is about 3400 Da and other sizes of PEG can also be used. The modified protein A/G (MPAG) is still water soluble and can be added directly into cell culture. FIG. 1B shows the membrane anchor shown in FIG. 1A incorporated into the plasma cell membrane of the cell. Many such molecules can be incorporated into a single cell. FIG. 1C shows an antibody attached to (captured by) the protein A/G portion of the modified protein A/G. The antibody has been secreted from the antibody secreted cell to which the modified protein A/G is attached (e.g., CHO cells, hybridoma cells, B cells). A cell pool includes many different cells, and the antibodies produced by each of the cells can be attached to the membrane anchor on its respective cell. As indicated above, a generic antibody capture molecule is configured to capture antibodies, and may be able to capture antibodies from various species or classes.

A generic antibody capture molecule of a membrane anchor captures antibodies regardless of their epitope specificity. Thus, there may be no need for multiple forms of membrane anchors for assaying different populations of cells or different classes or subclasses of antibodies. Membrane anchors may be applied to a population of cells in a suitable way. For example, a cell population may be grown in cell media, washed, and treated or exposed to an appropriate concentration of a water-soluble presentation agent (e.g., a membrane anchor) in the fresh media. Excess or unbound presentation agent may be removed with a fresh change of cell media.

In any of the methods described herein, the cells may be exposed to the presentation agent may in a media (e.g., cell media) that is free (substantially free) of albumen and any other agent that may otherwise inhibit the incorporation of the lipid-modified presentation agent into the cell membrane (e.g., albumen, serine, etc.).

Once a cell has presentation agent attached to its surface, antibodies secreted from the cell can attach to the anchors on its surface. Since the attaching of the antibodies to the presentation agent is based on antibody structure (rather than the epitope), most or many cells in a population can have antibodies attached to them. The levels of actual antibody expressed may be difficult to assess manually. Thus, described herein are automated methods in which a single cell sorter may be used.

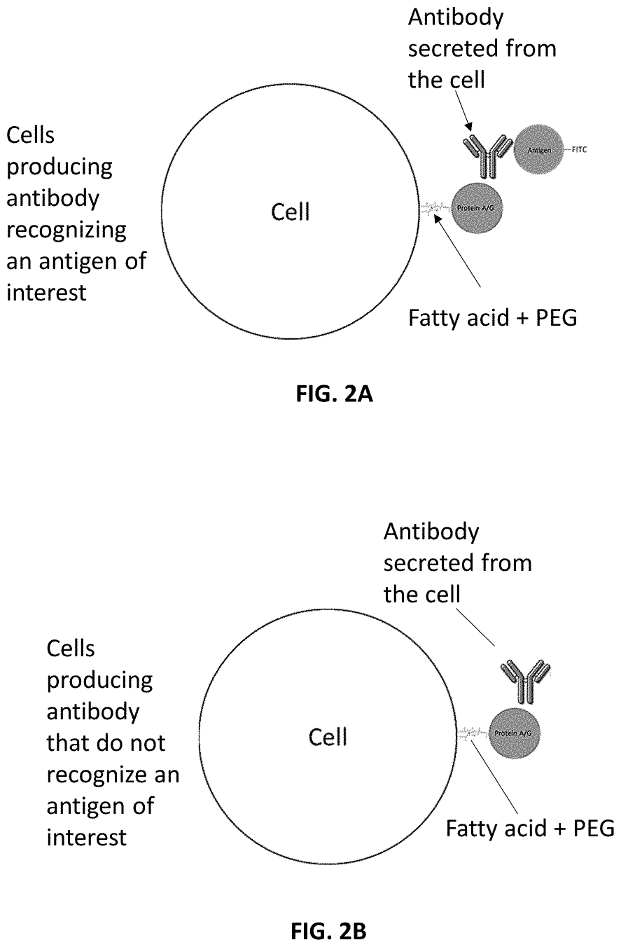

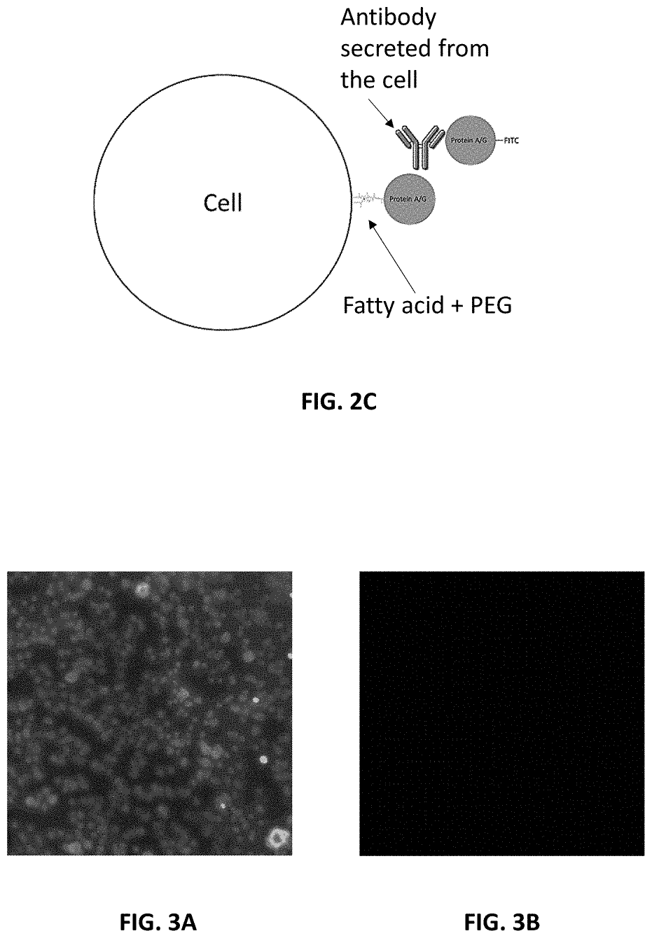

The cells can be subject to different types of assays, such as a specific antibody detection assay or a quantitative assay. In a specific antibody detection assay, the cells are assayed (e.g., with a specific detection agent) to determine which cell(s) out of many bind to a specific antigen. The level of antibody body production may also be assayed. In a quantitative assay, cells in a pool of cells may produce the same antibody and are assayed to determine which cells produce more or less, antibodies. For example, to perform a specific antibody detection assay, a specific antigen may be detectably labeled to use as a detectably labeled marker (e.g., fluorescently labeled, radioactively labeled) during the assay. FIG. 2A is a diagram showing secreted antibodies are captured by MPAG and detected by FITC conjugated antigen. The antigen binds to specific antibodies that recognize it (FIG. 2A). It will not bind to antibodies that do not recognize it (FIG. 2B).

For example, to perform a quantitative assay, a pool of cells may be treated with a antibody capture molecule that binds non-specifically to antibodies. A generic detection agent (e.g., a detectably labeled antibody capture molecule) can be, for example, protein A, protein G, or protein A/G that is detectably labeled (e.g., with a florescent label). It can be different from or the same as the generic antibody capture molecule. FIG. 2C is a diagram showing secreted antibodies are captured by MPAG and detected by FITC conjugated protein A/G (FPAG). In FIG. 2A the MPAG is incorporated into the cell plasma membrane. Because Protein A/G binds to antibody via its Fc region, antibodies secreted from the cell coated with MPAG will be captured on the cell surface and not secreted out into the medium. Cell surface captured antibodies can be detected by fluorescence labeled Protein A/G, such as FITC conjugated Protein A/G (FIG. 2C) or fluorescence labeled antigen, such as FITC conjugated antigen (FIG. 2A).

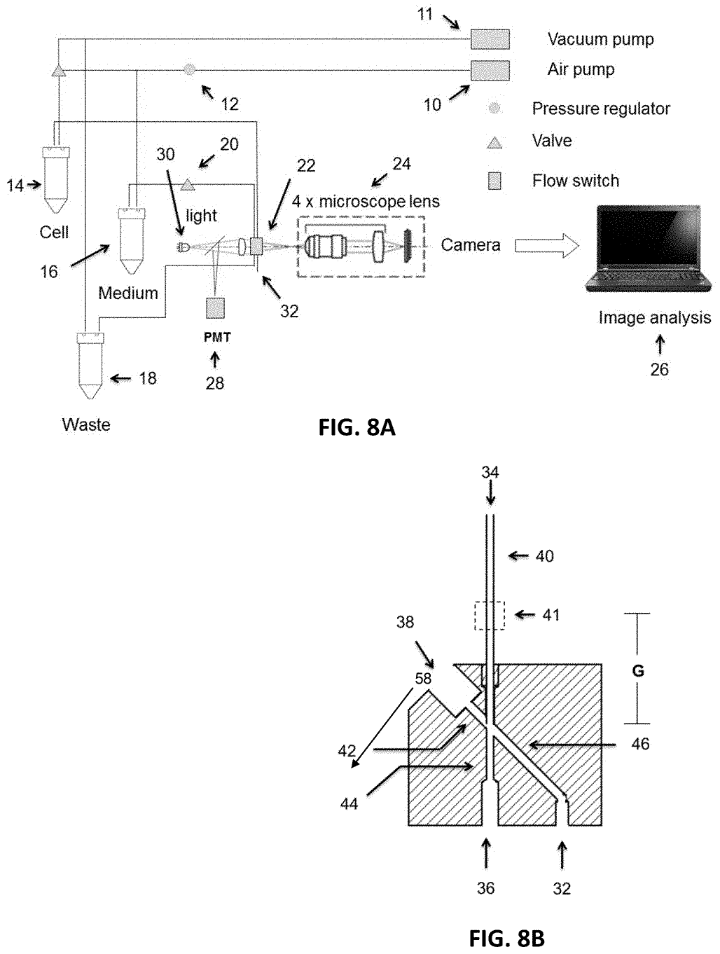

After binding a detectably labeled marker (a detectably labeled antigen marker or a detectably labeled antibody capture agent) is attached to the antibody of interest, the pool of cells may be assayed using a microparticle sorting and dispensing apparatus configured to analyze single cells such as a complex fluidic control flow cytometer or the apparatus described in U.S. Pat. No. 8,820,538. U.S. Pat. No. 8,820,538 describes apparatus (e.g., systems and devices) and methods for sorting microparticles for effectively and precisely sorting and dispensing individual microparticles, such as a single cell or small group of cells, to a very small area. These methods and apparatus do not require complex fluidic and control systems. U.S. Pat. No. 8,820,538 illustrates one example of a single cell sorter that utilizes flow switches that may be used for differentially sorting microparticles based on the flow rate of the microparticle and/or the flow rate of the fluid surrounding the microparticle. These flow switches differentially directs a fluid flowing through the flow switch based on the flow rate of the fluid. Further, these flow switches may be used in conjunction with an identification and control module which can determine when a microparticle having predetermined properties is within the flow switch, and can increase (or decrease) the flow rate of the fluid carrying the microparticle to sort the microparticle. As used herein, a flow switch is a switch that sorts a material between two (or more) outputs from the flow switch based on the flow rate of material as it passes through the flow switch. In particular, the flow switches may achieve differential flow sorting based on the differences between the resistance to flow through the outputs and different static fluid pressure at the interface between each output and the flow switch (e.g., the flow switch convergence region). FIG. 8A illustrates one example of a system that uses a flow switch to sort cells as described herein. FIG. 8B shows one example of a flow switch that may be used with the system of FIG. 8A.

For example, a flow switch typically operates by using at least two outlets connected to different outlet channels (e.g., a waste outlet flow path and a sample outlet flow path) exiting a convergence (e.g., intersection) region of the flow switch, where the different outlet flow paths have differential flow resistances and each outlet flow path has a different static fluid pressure. In some variations at low flow rates (e.g., flow rates below a lower threshold value) flow out of the flow switch will be through a first outlet flow path; at higher flow rates (e.g., flow rates above a higher threshold value), the flow out of the flow switch will be through a second outlet flow path. The devices may include visualization with microscope lens coupled with a digital camera to identify microparticles having a predetermined characteristic (e.g., size, shape, fluorescence or other marker, etc.). These devices may include optical light collecting system to measure fluorescent light emitted from microparticles.

In FIG. 8A, the cells (e.g., antibody-producing cells) may be stored in container (e.g., culture dish, plate, bottle, etc.) 14. The cells may be pre-mixed with the water-soluble presentation agent (including pre-incubating), or they may be mixed during the sorting procedure (immediately before sorting). The cells with water-soluble presentation agent may then be mixed or combined with the detection agent, either immediately before sorting, or some period before sorting. For example, in FIG. 8A, container 16 may contains a liquid, e.g., cell medium or saline buffer such as phosphate buffered saline; in some variations this liquid may include detection agent. Alternatively, the detection agent may be applied, incubated (e.g., 5 min, 10 min, 15 min, 20 min, 30 min, 40 min, 45 min, 1 hour etc. or less) and washed prior to sorting. Both containers 14, 16 may be pressurized by a micro-diaphragm gas pump 10. The pressure in container 14 and container 16 is regulated by pressure regulator 12. The pressure in container 14 and containers 16 may be 0-30 psi. In one embodiment, the pressure in container 14 and containers 16 is 2 psi. Container 14 is directly connected to one inlet of the flow switch 22 through silicone tube. When container 14 is pressurized, liquid in container 14 will constantly flow through silicone tube into flow switch 22. Container 16 is connected to the other inlet of flow switch 22 through silicone tube. The flow of liquid from container 16 to flow switch 22 is controlled by solenoid valve 20. When cells are flowed through the flow switch 22, they are detected through a detector. In this example, the detector includes a camera coupled with microscope lens 24; the use of a camera and/or a microscope lens is optional; alternatively just a florescent detector (e.g., PMT) may be used. For example, the fluorescence intensity of the cell may be measured by photomultiplier tube (PMT) 28. In some variations, if the cell does not meet the preset criteria, such as the size, shape and fluorescence intensity, solenoid valve 20 remains closed. The cell will flow out of flow switch into waste container 18. If the cell meets the preset criteria, solenoid valve 20 opens for short period of time. Thus, any of these methods and apparatuses may include immediately selecting and sorting cells based on their florescent intensity and therefore the expression level of the treated cells for the antibody.

In the example shown in FIG. 8A, medium will flow into flow switch 22. The majority of medium will flow out of sample channel 32. The flow of medium will carry the targeted cell out of the nozzle of the sample channel 32. Thus sorting and dispensing a single cell is achieved at the same time.

Successful sorting and dispensing cells in this example may depend on the specific design of this monolithic flow switch. Referring to the schematic illustration of FIG. 8B, in this example a flow switch has two flow inlets 34 and 38, connected to inlet flow paths 40 and 42, respectively, and two flow outlets 32 and 36, connected to the flow outlet paths 46 and 44, respectively, of the flow switch. The inlet and outlet flow paths all converge in a common convergence region 58. Inlet 34 is connected to container 14 and inlet 38 is connected to container 16. Microparticles flow into flow switch through a sample inlet flow path 40. Additional fluid flows through a flush inlet flow path 42 to alter the flow rate of fluid surrounding the microparticles in the flow switch from low flow rate to high flow rate. Outlet 32 is connected to sample channel and outlet 36 is connected to waste channel which leads to the waste container (container). The flow switch contains both microfluidic flow channels and macrofluidic flow channels. Sample inlet flow path 40 is a microfluidic channel. Flush inlet flow path 42, waste flow path 44 and sample outlet flow path 46 are macrofluidic channels. In one embodiment, sample inlet flow path 40 is made of glass capillary with rectangle cross-section with the dimension 30 μm×300 μm (H×W). In one embodiment, flush inlet flow path 42, waste flow path 44 and sample outlet flow path 46 are made from a single piece of polycarbonate. The cross-sections of flush inlet flow path 42, waste flow path 44 and sample outlet flow path 46 may be circular. In one embodiment, the diameters of flush inlet flow path 42 and sample outlet flow path 46 are 400 μm. The diameter of waste flow path 44 is 300 μm. Sample inlet flow path 40, flush inlet flow path 42, waste flow path 44 and sample outlet flow path 46 are converged at the center of flow switch.

To achieve cell sorting in this example, there must be as least two flow outlets: one for wanted (sample) cells and the other for unwanted (waste) cells. An easy way to change flow path between two flow outlets is to change the flow resistance between two flow outlets through valves. For example, there are valves A and B in the flow path A and B respectively. To let the liquid to flow only through flow path A, and not path B, simply turn valve A in the path A on and turn valve B in the path B off. However having two controllable valves in two flow path outlets creates large dead volume. This is why such a method is rarely used in cell sorting apparatus. Traditionally, cell sorting was achieved by keeping both flow outlet paths open and by applying certain amount of external physical forces, such as mechanical force, acoustic force, hydraulic force, optical forces, magnetic force, dielectrophoretic force, or electrostatic force as described in the background section, directly to a targeted cell to force it to move from one flow path to the other flow path. In contrast, in the flow switches described herein, both flow outlet paths are open, and no external force is used to switch flow paths. Switching between two flow paths may be achieved by simply changing flow rate into the flow switch.

While cells are flowed through the sample inlet flow path 40, they may be inspected (e.g., by a digital high speed camera) and their fluorescence intensities may be measured by PMT through an inspection window 41 (FIG. 2B). If the cell meets the preset criteria, such as size, shape and/or fluorescence intensity, valve 20 opens after a certain amount of delay, and medium flows into flow switch through a tube. The flow rate of medium into the flow switch is much larger than that of cell flow. In one embodiment, the medium flow rate is 500 ul/min. The diameter of silicone tube is larger than that of the waste channel. In one embodiment, the diameter of silicone tube is 0.762 mm whereas the diameter of silicone tube is 0.30 mm. The large flow through silicone tube into flow switch will change the flow pattern. A majority of medium will flow into sample outlet flow path 46 and out of sample channel because flow resistance through the sample channel is lower than that through waste channel. Movement of medium into sample outlet flow path 46 will also move targeted cell into sample outlet flow path 46 and out of the sample channel. Valve 20 only opens long enough so that the targeted could be dispensed out of the sample channel. In one embodiment, valve 20 opens for 25 ms. Thus single cell sorting and dispensing is achieved by changing the flow rate in the flow switch from 20 ul/min to 520 ul/min. Because target cell is dispensed out of the flow switch as a droplet through the sample channel 51, the location of dispensed droplet can be precisely controlled to accuracy less than 1 mm.

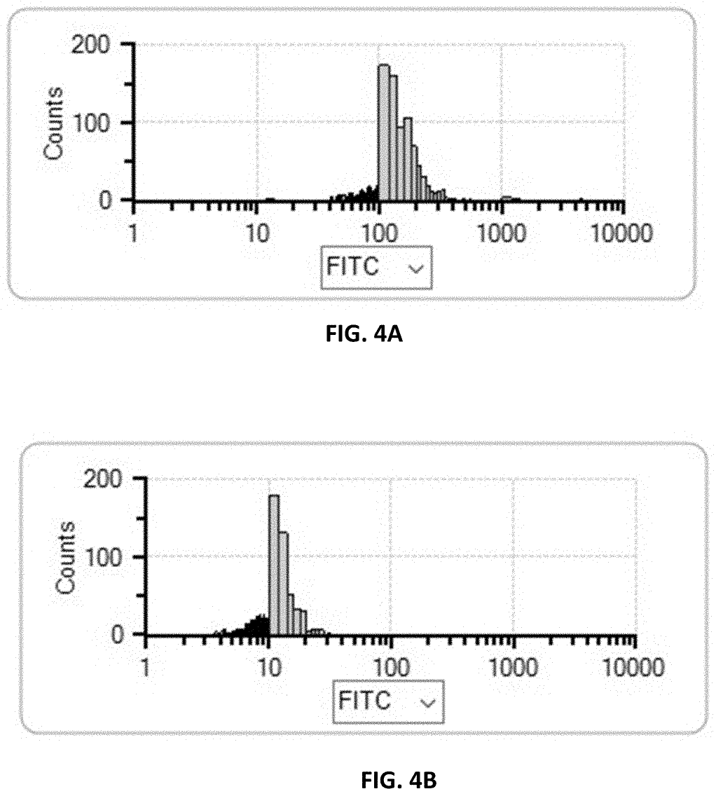

FIG. 3A shows a picture of CHO cells treated with MPAG (as shown in FIG. 1) and stained with FITC labeled mouse anti-human IgG antibody as described herein. FIG. 3B shows a picture of untreated CHO cells stained with FITC labeled mouse anti-human IgG antibody only. MPAG is incorporated into the cell plasma membrane. To qualified fluorescent intensity of MPAG treated cells stained with FITC conjugated mouse anti-human IgG antibodies, cells were analyzed with a microparticle sorting and dispensing apparatus, a Hana single cell dispenser (Namocell Inc, Mountain View Calif.). Cells showed fluorescent intensity around 100 (FIG. 4A). The negative control CHO cells showed fluorescent intensity around 10 (FIG. 4B).

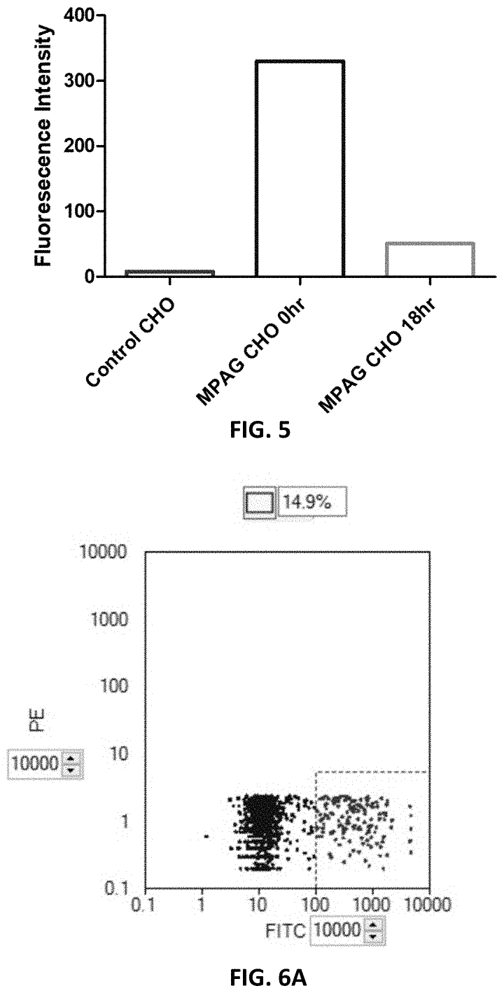

FIG. 5 shows results of an analysis of fluorescent intensity of CHO cells treated with MPAG and stained with FITC labeled mouse anti-human IgG antibody over time.

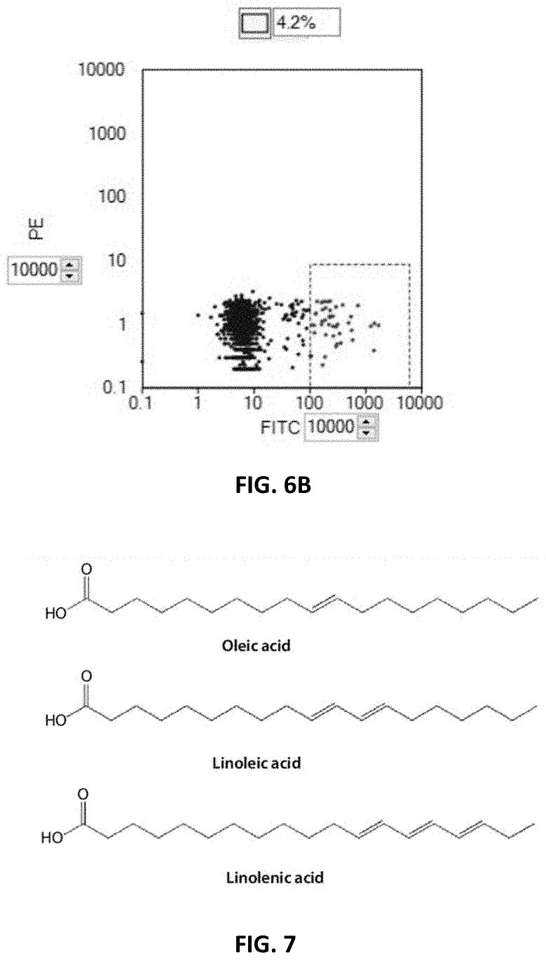

FIG. 6A shows the results of an analysis of CHO cells secreting a human IgG treated with MPAG and stained with FPAG. FIG. 6B shows the results of an analysis of CHO cells secreting a human IgG and treated with FPAG only.

In some variations, antibodies can be removed from captured cells such as by using a competing molecule. In general, sorted cells may be passaged and re-examined, either manually or automatically, to confirm a high level of expression of the target antibody.

### Experimental

MPAG is Incorporated into the Cell Plasma Membrane.

To test if MPAG can be readily incorporated into the cell plasma membrane, MPAG was added to 200 ul of CHO cells at 1×10⁶ cells/ml to a final concentration of 20 ug/ml. Cells were incubated at 37° C. for 20 minutes. Then cells were spun down at 300×g for 3 minutes and medium containing MPAG was removed and cells were resuspended in 200 ul of fresh medium. FITC conjugated mouse anti-human IgG antibodies were added into the cells and incubated at room temperature for 10 minutes. After 10 minutes, cells were spun down at 300×g for 3 minutes and were resuspended in 100 ul of fresh medium. Resuspended cells were checked under fluorescence microscopy. All the cells appeared to be green (FIG. 3A). As a negative control, CHO cells without MPAG treatment were stained with FITC conjugated mouse anti-human IgG antibodies. None of the cells were green (FIG. 3B). To qualify fluorescent intensity of MPAG treated cells stained with FITC conjugated mouse anti-human IgG antibodies, cells were analyzed with Hana single cell dispenser (Namocell Inc, Mountain View, Calif.). Cells showed fluorescent intensity around 100 (FIG. 4A). The negative control CHO cells showed fluorescent intensity around 10 (FIG. 4B).

Some MPAG Remains on the Plasma Membrane for a Period of Time.

To test how long MPAG would remain on the plasma membrane, 200 ul of CHO cells at 1×10⁶ cells/ml were labeled with 40 ug/ml of MPAG at 37° C. for 20 minutes. Cells were incubated at 37° C. for 20 minutes. Then cells were spun down at 300×g for 3 minutes and medium containing MPAG was removed and cells were resuspended in 200 ul of fresh medium. Half of the cells were stained immediately with FITC conjugated mouse anti-human IgG antibodies incubated at room temperature for 10 minutes. The other half were cultured at 37° C. for 18 hours and then stained with FITC conjugated mouse anti-human IgG antibodies. As a negative control, CHO cells without MPAG treatment were stained with FITC conjugated mouse anti-human IgG antibodies. Stained cells were analyzed with a Hana single cell dispenser (Namocell Inc, Mountain View, Calif.). FIG. 5 shows that the average fluorescent intensity of negative control is 8. The average fluorescent intensity of MPAG treated CHO cells immediately stained with FITC conjugated mouse anti-human IgG antibodies is 330. After 18 hours, some of MPAG remained on the plasma membrane, but a majority of the MPAG either internalized or diffused to the cell culture medium. The average fluorescent intensity of cells 18 hours after MPAG treatment is 51.

To test if MPAG can capture antibody secreted from the cell, CHO cells were transfected with a plasmid expressing a human antibody heavy chain and light chain. 200 ul of CHO cells at 1×10⁶ cells/ml from the transfection pool were spun down at 300×g for 3 minutes. Old medium was removed and 200 ul of fresh medium were added to the CHO cells. Cells were labeled with 40 ug/ml of MPAG at 37° C. for 20 minutes. After 20 minutes, cells were spin down at 300×g for 3 minutes and medium containing MPAG was removed and cells were resuspended in 200 ul of fresh medium and then the CHO cells were left in the cell culture incubator for 1 hour. During the 1 hour, the CHO cells secreted antibodies. The secreted antibodies were captured by MPAG on the cell surface. To detect secreted antibodies on the CHO cell surface, FPAG was added to the CHO cells after 1 hour to a final concentration of 40 ug/ml. The CHO cells were incubated at room temperature for 10 minutes. After 10 minutes, cells were spin down 300×g for 3 minutes and were resuspended in 200 ul of fresh medium. Cells were analyzed with a Hana single cell dispenser (Namocell Inc, Mountain View, Calif.). 14.9% of cells showed fluorescent intensity more than 100. As a negative control, 200 ul of CHO cell transfection pool without MPAG treatment were stained with 40 ug/ml FPAG at room temperature for 10 minutes. 4.2% cells showed fluorescent intensity more than 100. Because a transfection pool was used in this test, some of cells did not secrete antibody. This could explain why only 14.9% CHO cells showed fluorescent intensity more than 100. In the negative control, 4.2% of cells which were stained with FPAG were mostly likely dead cells or antibodies stuck on the plasma membrane due to defects in the secretion pathway.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of isolating an antibody producing cell, the method comprising:
contacting a population of antibody-producing cells with a soluble presentation construct comprising a lipid modified protein A molecule (MPA), lipid modified protein G molecule (MPG) or a lipid modified protein A/G molecule (MPAG) so that the MPA, MPG or MPAG is presented on the surface of the antibody-producing cells;
incubating the antibody-producing cells so that antibody produced by the cells is captured by the MPA, MPG or MPAG on the surface of the antibody-producing cells;
exposing the antibody-producing cells to a detection agent comprising a detectably labeled antigen, a detectably labeled protein A molecule, a detectably labeled protein G molecule and/or a detectably labeled protein A/G molecule;
sorting the cells using a cell sorting device according to the level of the detection agent.

2. The method of claim 1, wherein the concentration of the soluble presentation construct is less than 100 uM.

3. The method of claim 1, wherein the detection agent comprises a fluorescently-labeled protein A/G (FPAG).

4. The method of claim 1, wherein the detection agent comprises a fluorescently-labeled protein A construct (FPA) or a fluorescently-labeled protein G construct (PFG).

5. The method of claim 1, wherein the MPA, MPG and/or MPAG comprises a fatty acid based chain attached to a solubilizer.

6. The method of claim 1, wherein the MPA, MPG and/or MPAG comprises at least two fatty acid based chains attached to a solubilizer.

7. The method of claim 1, wherein the MPA, MPG and/or MPAG comprises at least two omega-9 fatty acid based chains attached to a solubilizer.

8. The method of claim 1, wherein the MPA, MPG and/or MPAG comprises at least two oleic acid based chains attached to a solubilizer.

9. The method of claim 1, wherein the solubilizer comprises an attached poly(ethylene glycol) (PEG) polymer.

10. The method of claim 1, wherein contacting a population of antibody-producing cells comprises contacting a hybridoma.

11. The method of claim 1, wherein contacting a population of antibody-producing cells comprises contacting a population of Chinese Hamster Ovary (CHO) cells.

12. The method of claim 1, wherein contacting a population of antibody-producing cells comprises contacting a population of antibody-producing B cells.

13. The method of claim 1, wherein contacting a population of antibody-producing cells comprises contacting a population of antibody-producing plasma cells.

14. The method of claim 1, wherein the soluble presentation construct consists essentially of MPAG.

15. The method of claim 1, wherein the soluble presentation construct consists essentially of MPA.

16. The method of claim 1, wherein the soluble presentation construct consists essentially of MPG.

17. The method of claim 1, wherein exposing the antibody-producing cells to the detection agent comprises exposing the antibody-producing cells to a detection agent having a detectably labeled marker comprising a fluorescently labeled antigen.

18. A method of isolating an antibody producing cell, the method comprising:
contacting a population of antibody-producing cells with a soluble presentation construct comprising a lipid modified protein A/G molecule (MPAG) having at least two oleic acid based chains coupled to a poly(ethylene glycol) (PEG) polymer that is coupled to the protein A/G molecule, so that the MPAG is presented on the surface of the antibody-producing cells;
incubating the antibody-producing cells so that antibody produced by a cell in the population of antibody-producing cells is captured by the MPAG on the surface of that cell;
exposing the antibody-producing cells to a detection agent detection agent comprising a detectably labeled marker that binds to the antibody produced by the cells;
sorting the cells using a cell sorting device according to the level of the detection agent.

19. The method of claim 18, wherein the detection agent comprises an antigen of interest coupled to the labeled marker, wherein the antibody binds to the antigen of interest.

20. The method of claim 18, wherein the detection agent comprises protein A/G coupled to a florescent marker.

21. The method of claim 18, wherein the incubating comprises incubating for between 1 minutes and 24 hours.

22. A kit for isolating antibody-producing cells, the kit comprising:
a water-soluble presentation agent comprising a solution of a lipid modified protein A/G molecule (MPAG) having at least two oleic acid based chains coupled to a poly(ethylene glycol) (PEG) polymer that is coupled to the protein A/G molecule; and
a detection agent comprising a solution of fluorescently labeled protein A/G.

23. A kit for isolating antibody-producing cells, the kit comprising:
a water-soluble presentation agent comprising a solution of a lipid modified protein A/G molecule (MPAG) having at least two oleic acid based chains coupled to a poly(ethylene glycol) (PEG) polymer that is coupled to the protein A/G molecule; and
a composition configured to attach a fluorescent label to an antigen protein.
